SEQUENCE LISTING (3 sequences)

SEQ ID NO: 1 - DNA, 2118 bp, Artificial Sequence
  codon-optimized rhesus monkey CEA
atgggcagccccagcgcccccctgcaccgctggtgcatcccctggcagaccctgctgctgaccgccagcctgctgaccttctggaacccccccaccaccgcccagctgaccatcgagagccgccccttcaacgtggccgagggcaaggaggtgctgctgctggcccacaacgtgagccagaacctgttcggctacatctggtacaagggcgagcgcgtggacgccagccgccgcatcggcagctgcgtgatccgcacccagcagatcacccccggccccgcccacagcggccgcgagaccatcgacttcaacgccagcctgctgatccacaacgtgacccagagcgacaccggcagctacaccatccaggtgatcaaggaggacctggtgaacgaggaggccaccggccagttccgcgtgtaccccgagctgcccaagccctacatcagcagcaacaacagcaaccccgtggaggacaaggacgccgtggccctgacctgcgagcccgagacccaggacaccacctacctgtggtgggtgaacaaccagagcctgcccgtgagcccccgcctggagctgagcagcgacaaccgcaccctgaccgtgttcaacatcccccgcaacgacaccaccagctacaagtgcgagacccagaaccccgtgagcgtgcgccgcagcgaccccgtgaccctgaacgtgctgtacggccccgacgcccccaccatcagccccctgaacaccccctaccgcgccggcgagaacctgaacctgacctgccacgccgccagcaaccccaccgcccagtacttctggttcgtgaacggcaccttccagcagagcacccaggagctgttcatccccaacatcaccgtgaacaacagcggcagctacatgtgccaggcccacaacagcgccaccggcctgaaccgcaccaccgtgaccgccatcaccgtgtacgccgagctgcccaagccctacatcaccagcaacaacagcaaccccatcgaggacaaggacgccgtgaccctgacctgcgagcccgagacccaggacaccacctacctgtggtgggtgaacaaccagagcctgagcgtgagcagccgcctggagctgagcaacgacaaccgcaccctgaccgtgttcaacatcccccgcaacgacaccaccttctacgagtgcgagacccagaaccccgtgagcgtgcgccgcagcgaccccgtgaccctgaacgtgctgtacggccccgacgcccccaccatcagccccctgaacaccccctaccgcgccggcgagaacctgaacctgagctgccacgccgccagcaaccccgccgcccagtacagctggttcgtgaacggcaccttccagcagagcacccaggagctgttcatccccaacatcaccgtgaacaacagcggcagctacatgtgccaggcccacaacagcgccaccggcctgaaccgcaccaccgtgaccgccatcaccgtgtacgtggagctgcccaagccctacatcagcagcaacaacagcaaccccatcgaggacaaggacgccgtgaccctgacctgcgagcccgtggccgagaacaccacctacctgtggtgggtgaacaaccagagcctgagcgtgagcccccgcctgcagctgagcaacggcaaccgcatcctgaccctgctgagcgtgacccgcaacgacaccggcccctacgagtgcggcatccagaacagcgagagcgccaagcgcagcgaccccgtgaccctgaacgtgacctacggccccgacacccccatcatcagcccccccgacctgagctaccgcagcggcgccaacctgaacctgagctgccacagcgacagcaaccccagcccccagtacagctggctgatcaacggcaccctgcgccagcacacccaggtgctgttcatcagcaagatcaccagcaacaacagcggcgcctacgcctgcttcgtgagcaacctggccaccggccgcaacaacagcatcgtgaagaacatcagcgtgagcagcggcgacagcgcccccggcagcagcggcctgagcgcccgcgccaccgtgggcatcatcatcggcatgctggtgggcgtggccctgatgtga

SEQ ID NO: 2 - protein, 705 aa, Macaca Mulatta
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARATVGIIIGMLVGVALM

SEQ ID NO: 3 - protein, 705 aa, Macaca Mulatta
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYFWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNNGAYACFVSNLATGRNNSIVKNISVSSGDSAPGSSGLSARATVGIIIGMLVGVALM

(19) United States
(12) Patent Application Publication
(10) Pub. No.: US 2006/0286114 A1
(43) Pub. Date: Dec. 21, 2006

(54) Synthetic gene encoding rhesus monkey carcinoembryonic antigen and uses thereof

(76) Inventors: Luigi Aurisicchio, POMEZIA, ROME (IT); Nicola La Monica, Rome (IT); Patrizia Giannetti, Rome (IT); Gennaro Ciliberto, Rome (IT)

Correspondence Address: MERCK AND CO., INC, P O BOX 2000, RAHWAY, NJ, 07065-0907, US

(21) Appl. No.: 10/568,168

(22) PCT Filed: Aug. 17, 2004
(86) PCT No.: PCT/EP04/09239

§ 371 Date: Feb. 9, 2006

Related U.S. Application Data
(60) Provisional application No. 60/497,201, filed on Aug. 22, 2003.

(51) Int. Cl. A61K 39/00; C07H 21/04; C12P 21/06; C12N 5/06; C07K 14/82; A61K 48/00

(52) U.S. Cl. 424/185.1; 435/69.1; 435/362; 435/320.1; 530/350; 536/23.5; 514/44

(57) ABSTRACT

Synthetic polynucleotides encoding rhesus monkey carcinoembryonic antigen (CEA) are provided, the synthetic polynucleotides being condon-optimized for expression in a human cellular environment. The gene encoding CEA is commonly associated with the development of human carcinomas. The present invention provides compositions and methods to elicit or enhance immunity to the protein product expressed by the CEA tumor-associated antigen, wherein aberrant CEA expression is associated with a carcinoma or its development. This invention specifically provides adenovial vector and plasmid constructs carrying codon-optimed rhesus monkey CEA and discloses their use in vaccines and pharmaceutical compositions for preventing and treating cancer.

### FIELD OF THE INVENTION

The present invention relates generally to the therapy of cancer. More specifically, the present invention relates to synthetic polynucleotides encoding the rhesus monkey homologue of the human tumor associated polypeptide carcinoembryonic antigen, herein designated rhCEAopt, wherein the polynucleotides are codon-optimized for expression in a human cellular environment. The present invention also provides recombinant vectors and hosts comprising said synthetic polynucleotides. This invention also relates to adenoviral vector and plasmid constructs carrying rhCEAopt and to their use in vaccines and pharmaceutical compositions for preventing and treating cancer.

### BACKGROUND OF THE INVENTION

The immunoglobulin superfamily (IgSF) consists of numerous genes that code for functionally diverse proteins. One important function of IgSF proteins is intercellular adhesion. IgSF proteins contain at least one Ig-related domain that is important for maintaining proper intermolecular binding interactions. Because such interactions are necessary to the diverse biological functions of the IgSF members, disruption or aberrant expression of many IgSF adhesion molecules has been correlated with human disease.

The carcinoembryonic antigen (CEA) belongs to a subfamily of the IgSF consisting of cell surface glycoproteins. Members of the CEA subfamily are known as CEA-related cell adhesion molecules (CEACAMs). The gene that encodes the CEA protein is often referred to as CEACAM5. Functionally, CEACAMs have been shown to act as both homotypic and heterotypic intercellular adhesion molecules (Benchimol et al., Cell 57:327-334 (1989)). In addition to cell adhesion, CEA inhibits cell death resulting from detachment of cells from the extracellular matrix and contributes to cellular transformation associated with certain proto-oncogenes such as Bcl2 and C-Myc (see Berinstein, J. Clin Oncol. 20(8): 2197-2207 (2002)).

Sequences coding for wild-type human CEA have been cloned and characterized (U.S. Pat. No. 5,274,087;U.S. Pat. No. 5,571,710; and U.S. Pat. No. 5,843,761. See also Beauchemin et al., Mol. Cell. Biol. 7:3221-3230 (1987); Zimmerman et al., Proc. Natl. Acad. Sci. USA 84:920-924 (1987); Thompson et al. Proc. Natl. Acad. Sci. USA 84(9):2965-69 (1987)).

Expression of CEA is normally detected during fetal development and in adult colonic mucosa. Overexpression of CEA is commonly associated with various malignancies. Such overexpression was first detected in human colon tumors over thirty years ago (Gold and Freedman, J. Exp. Med. 121:439-462 (1965)), and has since been found in nearly all colorectal tumors. Additionally, CEA overexpression is detectable in a high percentage of adenocarcinomas of the pancreas, breast and lung. Because of the prevalence of CEA expression in these tumor types, CEA is widely used clinically in the management and prognosis of these cancers.

The correlation between CEA expression and metastatic growth has led to its identification as a target for molecular and immunological intervention for colorectal cancer treatment. One therapeutic approach targeting CEA is the use of anti-CEA antibodies (see Chester et al., Cancer Chemother. Pharmacol. 46 (Suppl): S8-S12 (2000)). Another approach is the activation of the immune system to attack CEA-expressing tumors using CEA-based vaccines (for review, see Berinstein, supra). However, because CEA is a normal self-component that is overexpressed in cancer cells, specific immunotherapy targeting CEA must overcome self-tolerance.

The development and commercialization of many vaccines have been hindered by difficulties associated with obtaining high expression levels of exogenous genes in successfully transformed host organisms. Therefore, despite the identification of the wild-type nucleotide sequences encoding CEA proteins described above, it would be highly desirable to develop a readily renewable source of CEA protein that utilizes CEA-encoding nucleotide sequences that are optimized for expression in the intended host cell, said source allowing for the development of a cancer vaccine which is efficacious and not hindered by self-tolerance.

### SUMMARY OF THE INVENTION

The present invention relates to compositions and methods to elicit or enhance immunity to the protein products expressed by CEA genes, which have been associated with numerous adenocarcinomas, including colorectal carcinomas. Specifically, the present invention provides polynucleotides encoding rhesus monkey CEA protein, wherein said polynucleotides are codon-optimized for high level expression in a human cell. The present invention further provides adenoviral and plasmid-based vectors comprising the synthetic polynucleotides and discloses use of said vectors in immunogenic compositions and vaccines for the prevention and/or treatment of CEA-associated cancer.

The present invention also relates to synthetic nucleic acid molecules (polynucleotides) comprising a sequence of nucleotides that encode rhesus monkey carcinoembryonic antigen (hereinafter rhCEA) as set forth in SEQ ID NO:2 or SEQ ID NO:3, wherein the synthetic nucleic acid molecules are codon-optimized for high-level expression in a human cell (hereinafter rhCEAopt). The nucleic acid molecules disclosed herein may be transfected into a host cell of choice wherein the recombinant host cell provides a source for substantial levels of an expressed functional rhCEA protein (SEQ ID NOs:2 and 3).

The present invention further relates to a synthetic nucleic acid molecule which encodes mRNA that expresses a rhesus monkey CEA protein; this DNA molecule comprising the nucleotide sequence disclosed herein as SEQ ID NO:1. A preferred aspect of this portion of the present invention is disclosed in FIG. 1, which shows a DNA molecule (SEQ ID NO:1) that encodes a rhCEA protein (SEQ ID NO:2). The preferred nucleic acid molecule of the present invention is codon-optimized for high-level expression in a human cell.

The present invention also relates to recombinant vectors and recombinant host cells, both prokaryotic and eukaryotic, which contain the nucleic acid molecules disclosed throughout this specification.

The present invention further relates to a process for expressing a codon-optimized rhesus monkey CEA protein in a recombinant host cell, comprising: (a) introducing a vector comprising a nucleic acid molecule that encodes rhesus monkey CEA protein into a suitable host cell, wherein the nucleic acid molecule is codon-optimized for optimal expression in the host cell; and, (b) culturing the host cell under conditions which allow expression of said codon-optimized rhesus monkey protein.

Another aspect of this invention is a method of preventing or treating cancer comprising administering to a mammal a vaccine vector comprising a synthetic nucleic acid molecule, the synthetic nucleic acid molecule comprising a sequence of nucleotides that encodes a rhesus monkey carcinoembryonic antigen (rhCEA) protein as set forth in SEQ ID NO:2 or as set forth in SEQ ID NO:3, wherein the synthetic nucleic acid molecule is codon-optimized for high level expression in a human cell.

The present invention further relates to an adenovirus vaccine vector comprising an adenoviral genome with a deletion in the E1 region, and an insert in the E1 region, wherein the insert comprises an expression cassette comprising: (a) a codon-optimized polynucleotide encoding a rhesus monkey CEA protein; and (b) a promoter operably linked to the polynucleotide.

The present invention also relates to a vaccine plasmid comprising a plasmid portion and an expression cassette portion, the expression cassette portion comprising: (a) a synthetic polynucleotide encoding a rhesus monkey CEA protein, wherein the synthetic polynucleotide is codon-optimized for optimal expression in a human cell; and (b) a promoter operably linked to the polynucleotide.

Another aspect of the present invention is a method of protecting a mammal from cancer or treating a mammal suffering from CEA-associated cancer comprising: (a) introducing into the mammal a first vector comprising: i) a codon-optimized polynucleotide encoding a rhesus monkey CEA protein; and ii) a promoter operably linked to the polynucleotide; (b) allowing a predetermined amount of time to pass; and (c) introducing into the mammal a second vector comprising: i) a codon-optimized polynucleotide encoding a rhesus monkey CEA protein; and ii) a promoter operably linked to the polynucleotide.

As used throughout the specification and in the appended claims, the singular forms “a,” “an,” and “the” include the plural reference unless the context clearly dictates otherwise.

As used throughout the specification and appended claims, the following definitions and abbreviations apply:

The term “promoter” refers to a recognition site on a DNA strand to which the RNA polymerase binds. The promoter forms an initiation complex with RNA polymerase to initiate and drive transcriptional activity. The complex can be modified by activating sequences termed “enhancers” or inhibiting sequences termed “silencers”.

The terrn “cassette” refers to the sequence of the present invention that contains the nucleic acid sequence which is to be expressed. The cassette is similar in concept to a cassette tape; each cassette has its own sequence. Thus by interchanging the cassette, the vector will express a different sequence. Because of the restriction sites at the 5′ and 3′ ends, the cassette can be easily inserted, removed or replaced with another cassette.

The term “vector” refers to some means by which DNA fragments can be introduced into a host organism or host tissue. There are various types of vectors including plasmid, virus (including adenovirus), bacteriophages and cosmids.

The term “first generation,” as used in reference to adenoviral vectors, describes said adenoviral vectors that are replication-defective. First generation adenovirus vectors typically have a deleted or inactivated E1 gene region, and preferably have a deleted or inactivated E3 gene region.

The designation “pV1J-rhCEAopt” refers to a plasmid construct, disclosed herein, comprising the human CMV immediate-early (IE) promoter with intron A, a full-length codon-optimized human CEA gene, bovine growth hormone-derived polyadenylation and transcriptional termination sequences, and a minimal pUC backbone (see EXAMPLE 2). The designation “pV1J-rhCEA” refers to a construct as described above, except the construct comprises a wild-type rhesus monkey CEA gene instead of a codon-optimized rhesus monkey CEA gene.

The designations “MRKAd5/rhCEAopt” and “MRKAd5/rhCEA” refer to two constructs, disclosed herein, which comprise an Ad5 adenoviral genome deleted of the E1 and E3 regions. In the “MRKAd5/rhCEAopt” construct, the E1 region is replaced by a codon-optimized rhesus monkey CEA gene in an E1 parallel orientation under the control of a human CMV promoter without intron A, followed by a bovine growth hormone polyadenylation signal. The “MRKAd5rhCEA” construct is essentially as described above, except the E1 region of the AdS genome is replaced with a wild-type rhesus monkey CEA sequence (see EXAMPLE 2).

The term “effective amount” means sufficient vaccine composition is introduced to produce the adequate levels of the polypeptide, so that an immune response results. One skilled in the art recognizes that this level may vary.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutainic acid for aspartic acid).

“rhCEA” and “rbCEAopt” refer to a rhesus monkey carcinoembryonic antigen and a rhesus monkey codon-optimized carcinoembryonic antigen, respectively.

The term “mammalian” refers to any mammal, including a human being.

The abbreviation “Ag” refers to an antigen.

The abbreviations “Ab” and “mAb” refer to an antibody and a monoclonal antibody, respectively.

The abbreviation “ORF” refers to the open reading frame of a gene.

### BRIEF DESCRIPTION OF THE DRAWINGS

FIG. 1(A) shows the nucleotide sequence of a codon optimized rhesus monkey CEA cDNA (SEQ ID NO:1). See EXAMPLE 1. Panel (B) shows the predicted amino acid sequences of rhesus monkey CEA protein deduced from wild-type CEA nucleotide sequences isolated from two different rhesus monkeys (SEQ ID NOs:2 and 3). Differences between the two rhesus monkey amino acid sequences are indicated.

FIG. 2 shows a comparison between wild-type rhesus CEA expression and codon-optimized rhesus CEA expression, as determined by Western blot analysis. HeLa cells were either transfected with pV1J vector or infected with Ad5 expressing rhCEA or rhCEAopt at the indicated doses. Rhesus CEA was detected as a 180-200 KDa band.

FIG. 3 shows a comparison of rhCEA and rhCEAopt expression in C57BL/6 mice by ELISA. Mice were injected intramuscularly with Ad5 vectors at the indicated doses. Expression of circulating rhCEA in Ad5 injected mice was detected by ELISA 3 days later. Symbols represent OD₄₀₅ values for each single mouse of the group. The filled circles represent the geometric mean of each group.

FIG. 4 shows a comparison of the cellular immune response in C57BL/6 mice vaccinated with rhCEA or rhCEAopt-expressing vectors. Mice were immunized once with DNA or Ad5 at the indicated doses. Two weeks later, an ELISPOT assay was performed to measure the cell mediated immune response.

FIG. 5 depicts the humoral immune response in CEA transgenic (CEA.Tg) mice after four DNA injections. CEA.Tg mice were immunized 4 times with the indicated plasmid DNA, expressing either human or rhesus CEA. The two groups on the far right were immunized either with a 50% mix of rhesus and human CEAopt vectors or 3 times with rhCEAopt and lastly with hCEAopt. Total IgG and IgG isotypes were measured by ELISA.

FIG. 6 depicts the humoral immune response in CEA.Tg mice after four injections with the indicated DNA and one Ad5 injection. IgG titer was measured before (day 41) and after (day 57) Ad5 boosting. Total IgG and IgG isotypes were measured by ELISA.

FIG. 7 shows the cellular immune response in CEA.Tg mice immunized by DNA-Ad5 mixed modality. Immunizations were performed with the indicated combinations. Cellular immune response of groups of mice comprising 3 to 4 mice/group was determined by ELISPOT assay using peptide pools A, B, C, and D as stimulators. CD8 peptide is contained in pool D.

FIG. 8 shows that different epitopes of rhesus CEA are able to elicit a cellular immune response upon CEA.Tg mice immunization. The first column lists specific CEA peptides used. Results are reported as the number of spot forming colonies (SFC) per 10⁶ cells. Significant numbers of SFC measured by ELISPOT are indicated in bold. CD4+ and CD8+ epitopes, as determined by intracellular staining, are shown in solid gray and hatched gray cells, respectively. Epitopes that are able to activate both CD4+ and CD8+ IFNγ secretion are also indicated in dotted cells.

FIG. 9 shows expression of rhCEA in HeLa cells infected with Ad5 or Ad24 expressing rhCEAopt at the indicated doses. Rhesus CEA was detected as a 180-200 KDa band.

FIG. 10 shows a comparison of expression of rhCEAopt and rhCEA in CEA.Tg mice immunized with the indicated Ad vectors. Mice were injected intramuscularly with Ad5, Ad6 and Ad24 vectors at the dose of 1×10¹⁰ pp. Expression of circulating rhCEA was detected by ELISA 3 days after the injection. Each symbol represents expression data from a single mouse.

FIG. 11 shows the cellular immune response in CEA.Tg mice injected with various prime/boost modalities. Mice were injected twice at 1×10¹⁰ pp of adenoviral vectors with the indicated modality. The resulting immune response was measured by intracellular staining (ICS) on PBMC. Results for different peptide pools are expressed as a percentage of IFNγ+ cells.

FIG. 12 shows results of rhesus monkey immunization protocol CV-1, in which Ad24rhCEAopt was used to boost the immune response elicited by DNA and Ad5-rhCEA. Panel (A) depicts the specific immunization schedule. Panels (B and C) show the immune response for the indicated peptide pools for two different rhesus monkeys, measured by ELISPOT assay.

FIG. 13 shows the results of rhesus monkey immunization protocol CV-2, in which Ad5 and Ad24-rhCEAopt were used to boost the immune response elicited by hCEAopt expressing DNA. Panel (A) depicts the specific immunization schedule. Panels (B and C) show the immune response for the indicated peptide pool for two different rhesus monkeys, measured by ELISPOT assay.

FIG. 14 shows the results of rhesus monkey immunization protocol CV-3, in which CV33-rhCEAopt was used to boost the immune response elicited by Ad5-rhCEAopt. Panel (A) depicts the specific immunization schedule. Panel (B) shows the immune response for the indicated peptide pools for two different rhesus monkeys, measured by ELISPOT assay.

### DETAILED DESCRIPTION OF THE INVENTION

Carcinoembryonic antigen (CEA) is commonly associated with the development of adenocarcinomas. The present invention relates to compositions and methods to elicit or enhance immunity to the protein product expressed by the CEA tumor-associated antigen, wherein aberrant CEA expression is associated with the carcinoma or its development. Association of aberrant CEA expression with a carcinoma does not require that the CEA protein be expressed in tumor tissue at all timepoints of its development, as abnormal CEA expression may be present at tumor initiation and not be detectable late into tumor progression or vice-versa.

To this end, synthetic DNA molecules encoding the rhesus monkey CEA protein are provided. The codons of the synthetic molecules are designed so as to use the codons preferred by the projected host cell, which in preferred embodiments is a human cell. The synthetic molecules may be used for the development of recombinant adenovirus or plasmid-based vaccines, which provide effective immunoprophylaxis against CEA-associated cancer through neutralizing antibody and cell-mediated immunity. The synthetic molecules may be used as an immunogenic composition. This invention provides polynucleotides which, when directly introduced into a vertebrate in vivo, including mammals such as primates and humans, induce the expression of encoded proteins within the animal.

The present invention provides synthetic DNA molecules encoding the rhesus monkey CEA protein. The synthetic molecules of the present invention comprise a sequence of nucleotides, wherein some of the nucleotides have been altered so as to use the codons preferred by a human cell, thus allowing for high-level expression of rhCEA in a human host cell. The synthetic molecules may be used in a cancer vaccine to provide effective immunoprophylaxis against CEA-associated carcinomas through neutralizing antibody and cell-mediated immunity, or as a source of rhesus CEA protein.

A “triplet” codon of four possible nucleotide bases can exist in over 60 variant forms. Because these codons provide the message for only 20 different amino acids (as well as transcription initiation and termination), some amino acids can be coded for by more than one codon, a phenomenon known as codon redundancy. For reasons not completely understood, alternative codons are not uniformly present in the endogenous DNA of differing types of cells. Indeed, there appears to exist a variable natural hierarchy or “preference” for certain codons in certain types of cells. As one example, the amino acid leucine is specified by any of six DNA codons including CTA, CTC, CTG, CTT, TTA, and TTG. Exhaustive analysis of genome codon frequencies for microorganisms has revealed endogenous DNA of E. coli most commonly contains the CTG leucine-specifying codon, while the DNA of yeasts and slime molds most commonly includes a TTA leucine-specifying codon. In view of this hierarchy, it is generally believed that the likelihood of obtaining high levels of expression of a leucine-rich polypeptide by an E. coli host will depend to some extent on the frequency of codon use. For example, it is likely that a gene rich in TTA codons will be poorly expressed in E. colo, whereas a CTG rich gene will probably be highly expressed in this host. Similarly, a preferred codon for expression of a leucine-rich polypeptide in yeast host cells would be TTA.

The implications of codon preference phenomena on recombinant DNA techniques are manifest, and the phenomenon may serve to explain many prior failures to achieve high expression levels of exogenous genes in successfully transformed host organisms—a less “preferred” codon may be repeatedly present in the inserted gene and the host cell machinery for expression may not operate as efficiently. This phenomenon suggests that synthetic genes which have been designed to include a projected host cell's preferred codons provide an optimal form of foreign genetic material for practice of recombinant DNA techniques. Thus, one aspect of this invention is a human CEA gene that is codon-optimized for expression in a human cell. In a preferred embodiment of this invention, it has been found that the use of alternative codons encoding the same protein sequence may remove the constraints on expression of exogenous CEA protein in human cells.

In accordance with this invention, the rhesus monkey CEA gene sequence was converted to a polynucleotide sequence having an identical translated sequence but with alternative codon usage as described by Lathe, “Synthetic Oligonucleotide Probes Deduced from Amino Acid Sequence Data: Theoretical and Practical Considerations” J. Molec. Biol. 183:1-12 (1985), which is hereby incorporated by reference. The methodology generally consists of identifying codons in the wild-type sequence that are not commonly associated with highly expressed human genes and replacing them with optimal codons for high expression in human cells. The new gene sequence is then inspected for undesired sequences generated by these codon replacements (e.g., “ATTTA” sequences, inadvertent creation of intron splice recognition sites, unwanted restriction enzyme sites, high GC content, etc.). Undesirable sequences are eliminated by substitution of the existing codons with different codons coding for the same amino acid. The synthetic gene segments are then tested for improved expression.

The methods described above were used to create synthetic gene sequences for rhesus monkey CEA, resulting in a gene comprising codons optimized for high level expression in a human cell. While the above procedure provides a summary of our methodology for designing codon-optimized genes for use in cancer vaccines, it is understood by one skilled in the art that similar vaccine efficacy or increased expression of genes may be achieved by minor variations in the procedure or by minor variations in the sequence. One of skill in the art will also recognize that additional DNA molecules may be constructed that provide for high levels of rhesus monkey CEA expression in human cells, wherein only a portion of the codons of the DNA molecules are codon-optimized.

Accordingly, the present invention relates to a synthetic polynucleotide comprising a sequence of nucleotides encoding a rhesus monkey CEA protein (SEQ ID NOs:2 or 3), or a biologically active fragment or mutant form of a rhesus monkey CEA protein, the polynucleotide sequence comprising codons optimized for expression in a human host. Said mutant forms of the rhCEA protein include, but are not limited to: conservative amino acid substitutions, amino-terminal truncations, carboxy-terminal truncations, deletions, or additions. Any such biologically active fragment and/or mutant will encode either a protein or protein fragment which at least substantially mimics the immunological properties of the rhCEA protein as set forth in SEQ ID NO:2 and SEQ ID NO:3. The synthetic polynucleotides of the present invention encode mRNA molecules that express a functional rhesus monkey CEA protein so as to be useful in the development of a therapeutic or prophylactic cancer vaccine.

The present invention relates to a synthetic nucleic acid molecule (polynucleotide) comprising a sequence of nucleotides which encodes mRNA that expresses a novel rhCEA protein as set forth in SEQ ID NO:2 and SEQ ID NO:3, wherein the synthetic nucleic acid molecule is codon-optimized for high-level expression in a human host cell. The nucleic acid molecules of the present invention are substantially free from other nucleic acids.

The present invention also relates to recombinant vectors and recombinant host cells, both prokaryotic and eukaryotic, which contain the nucleic acid molecules disclosed throughout this specification. The synthetic DNA molecules, associated vectors, and hosts of the present invention are useful for the development of a cancer vaccine.

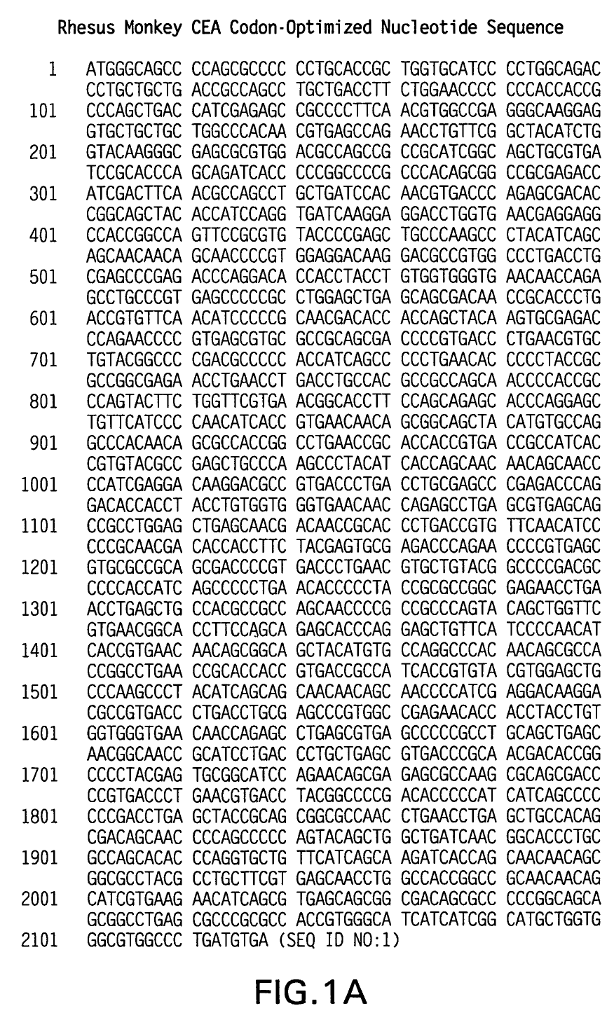

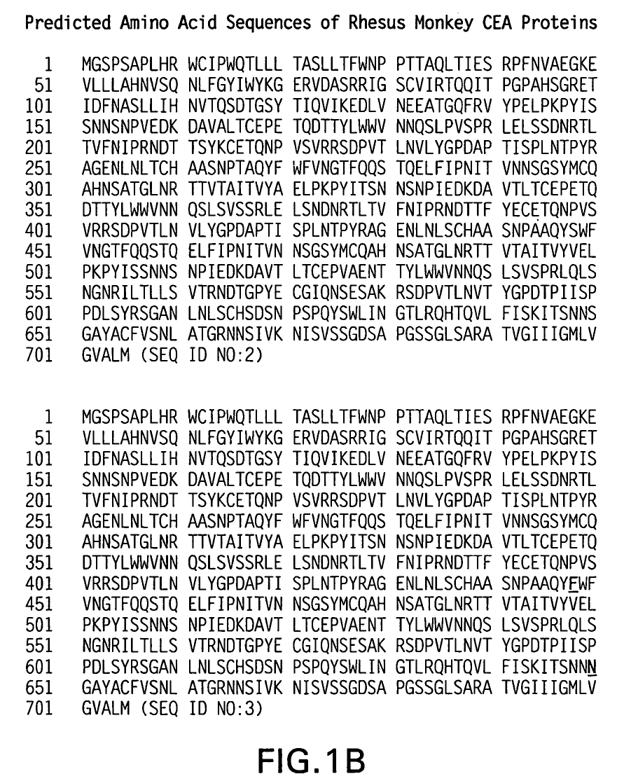

A preferred DNA molecule of the present invention comprises the nucleotide sequence disclosed herein as SEQ ID NO:1, shown in FIG. 1(A), which encodes the rhesus monkey CEA protein shown in FIG. 1(B) (top) and set forth as SEQ ID NO:2.

The present invention also includes biologically active fraginents or mutants-of SEQ ID NO: 1, which encode mRNA expressing rhesus monkey CEA proteins. Any such biologically active fragment and/or mutant will encode either a protein or protein fragment which at least substantially mimics the pharmacological properties of the rhCEA protein, including but not limited to the rhCEA proteins as set forth in SEQ ID NO:2 and SEQ ID NO:3. Any such polynucleotide includes but is not necessarily limited to: nucleotide substitutions, deletions, additions, amino-terminal truncations and carboxy-terminal truncations. The mutations of the present invention encode mRNA molecules that express a functional rhCEA protein in a eukaryotic cell so as to be useful in cancer vaccine development.

This invention also relates to synthetic codon-optimized DNA molecules that encode the rhCEA protein wherein the nucleotide sequence of the synthetic DNA differs significantly from the nucleotide sequence of SEQ ID NO:1, but still encodes the rhCEA protein as set forth in SEQ ID NO:2 or the rhCEA protein set forth in SEQ ID NO:3. Also included within the scope of this invention are mutations in the DNA sequence that do not substantially alter the ultimate physical properties of the expressed protein. For example, substitution of valine for leucine, arginine for lysine, or asparagine for glutamine may not cause a change in the functionality of the polypeptide.

It is known that DNA sequences coding for a peptide may be altered so as to code for a peptide that has properties that are different than those of the naturally occurring peptide. Methods of altering the DNA sequences include but are not limited to site directed mutagenesis. Examples of altered properties include but are not limited to changes in the affinity of an enzyme for a substrate or receptor for a ligand.

The present invention also relates to rhCEAopt fusion constructs, including but not limited to fusion constructs which express a portion of the rhesus monkey CEA protein linked to various markers, including but in no way limited to GFP (Green fluorescent protein), the MYC epitope, GST, and Fc. Any such fusion construct may be expressed in the cell line of interest and used to screen for modulators of the rhesus monkey CEA protein disclosed herein. Also contemplated are fusion constructs that are constructed to enhance the immune response to rhesus monkey CEA including, but not limited to: DOM and hsp70, and LTB.

The present invention further relates to recombinant vectors that comprise the synthetic nucleic acid molecules disclosed throughout this specification. These vectors may be comprised of DNA or RNA. For most cloning purposes, DNA vectors are preferred. Typical vectors include plasmids, modified viruses, baculovirus, bacteriophage, cosmids, yeast artificial chromosomes, and other forms of episomal or integrated DNA that can encode a rhCEA protein. It is well within the purview of the skilled artisan to determine an appropriate vector for a particular gene transfer or other use.

An expression vector containing codon-optimized DNA encoding a rhCEA protein may be used for high-level expression of rhCEA in a recombinant host cell. Expression vectors may include, but are not limited to, cloning vectors, modified cloning vectors, specifically designed plasmids or viruses. Also, a variety of bacterial expression vectors may be used to express recombinant rhCEA in bacterial cells if desired. In addition, a variety of fungal cell expression vectors may be used to express recombinant rhCEA in fungal cells. Further, a variety of insect cell expression vectors may be used to express recombinant protein in insect cells.

The present invention also relates to host cells transformed or transfected with vectors comprising the nucleic acid molecules of the present invention. Depending on the host cell of choice, the nucleotide sequence may be altered to include codons preferred by said host for high-level gene expression. Recombinant host cells may be prokaryotic or eukaryotic, including but not limited to, bacteria such as E. coli, fungal cells such as yeast, mammalian cells including, but not limited to, cell lines of bovine, porcine, monkey and rodent origin; and insect cells including but not limited to Drosophila and silkworm derived cell lines. Such recombinant host cells can be cultured under suitable conditions to produce rhCEA or a biologically equivalent form. In a preferred embodiment of the present invention, the host cell is human. As defined herein, the term “host cell” is not intended to include a host cell in the body of a transgenic human being, human fetus, or human embryo.

As noted above, an expression vector containing DNA encoding a rhCEA protein may be used for expression of rhCEA in a recombinant host cell. Therefore, another aspect of this invention is a process for expressing a rhesus monkey CEA protein in a recombinant host cell, comprising: (a) introducing a vector comprising a nucleic acid that encodes rhesus monkey CEA protein into a suitable human host cell, wherein the rhesus monkey CEA protein comprises a sequence of amino acids as set forth in SEQ ID NO:2 or SEQ ID NO:3, and wherein the nucleic acid is codon-optimized for optimal expression in the host cell; and, (b) culturing the host cell under conditions which allow expression of said rhesus monkey CEA protein.

In a preferred embodiment of this aspect of the invention, the nucleic acid comprises a sequence of nucleotides as set forth in SEQ ID NO:1.

Following expression of rhCEA in a host cell, rhCEA protein may be recovered to provide rhCEA protein in active form. Several rhCEA protein purification procedures are available and suitable for use. Recombinant rhCEA protein may be purified from cell lysates and extracts by various combinations of, or individual application of salt fractionation, ion exchange chromatography, size exclusion chromatography, hydroxylapatite adsorption chromatography and hydrophobic interaction chromatography. In addition, recombinant rhCEA protein can be separated from other cellular proteins by use of an immunoaffinity column made with monoclonal or polyclonal antibodies specific for full-length rhCEA protein, or polypeptide fragments of rhCEA protein.

The nucleic acids of the present invention may be assembled into an expression cassette which comprises sequences designed to provide for efficient expression of the protein in a human cell. The cassette preferably contains a full-length codon-optimized rhCEA gene, with related transcriptional and translations control sequences operatively linked to it, such as a promoter, and termination sequences. In a preferred embodiment, the promoter is the cytomegalovirus promoter without the intron A sequence (CMV), although those skilled in the art will recognize that any of a number of other known promoters such as the strong immunoglobulin, or other eukaryotic gene promoters may be used. A preferred transcriptional terminator is the bovine growth hormone terminator, although other known transcriptional terminators may also be used. The combination of CMV-BGH terminator is particularly preferred.

In accordance with this invention, the rhCEAopt expression cassette is inserted into a vector. The vector is preferably an adenoviral vector, although linear DNA linked to a promoter, or other vectors, such as adeno-associated virus or a modified vaccinia virus, retroviral or lentiviral vector may also be used.

If the vector chosen is an adenovirus, it is preferred that the vector be a first-generation adenoviral vector. These adenoviral vectors are characterized by having a non-functional E1 gene region, and preferably a deleted adenoviral E1 gene region. In some embodiments, the expression cassette is inserted in the position where the adenoviral E1 gene is normally located. In addition, these vectors optionally have a non-functional or deleted E3 region. It is preferred that the adenovirus genome used be deleted of both the E1 and E3 regions (ΔE1ΔE3). The adenoviruses can be multiplied in known cell lines which express the viral E1 gene, such as 293 cells, or PERC.6 cells, or in cell lines derived from 293 or PERC.6 cell which are transiently or stablily transforrned to express an extra protein. For examples, when using constructs that have a controlled gene expression, such as a tetracycline regulatable promoter system, the cell line may express components involved in the regulatory system. One example of such a cell line is T-Rex-293; others are known in the art.

For convenience in manipulating the adenoviral vector, the adenovirus may be in a shuttle plasmid form. This invention is also directed to a shuttle plasmid vector which comprises a plasmid portion and an adenovirus portion, the adenovirus portion comprising an adenoviral genome which has a deleted E1 and optional E3 deletion, and has an inserted expression cassette comprising codon-optimized rhesus monkey CEA. In preferred embodiments, there is a restriction site flanking the adenoviral portion of the plasmid so that the adenoviral vector can easily be removed. The shuttle plasmid may be replicated in prokaryotic cells or eukaryotic cells.

In a preferred embodiment of the invention, the expression cassette is inserted into the pMRKAd5-HV0 adenovirus plasmid (See Emini et al., WO 02/22080, which is hereby incorporated by reference). This plasmid comprises an Ad5 adenoviral genome deleted of the E1 and E3 regions. The design of the pMRKAd5-HV0 plasmid was improved over prior adenovectors by extending the 5′ cis-acting packaging region further into the E1 gene to incorporate elements found to be important in optimizing viral packaging, resulting in enhanced virus amplification. Advantageously, this enhanced adenoviral vector is capable of maintaining genetic stability following high passage propagation.

Standard techniques of molecular biology for preparing and purifying DNA constructs enable the preparation of the adenoviruses, shuttle plasmids, and DNA immunogens of this invention.

It has been determined in accordance with the present invention that the synthetic cDNA molecule described herein (SEQ ID NO:1), which is codon-optimized for high-level expression in a human cell, is expressed with greater efficiency than the corresponding wild type sequence. In a CEA transgenic mouse model, xenogeneic immunization with the codon optimized cDNA of rhCEA breaks tolerance to rhCEA more efficiently than the wild type sequence. Additionally, it was shown herein that rhCEAopt is more immunogenic that rhCEA and is more efficient in eliciting both cellular and humoral immune responses. Further, rhCEAopt expressing vectors efficiently break the immune tolerance to rhCEA in rhesus monkeys, thus confirming the observations in mice.

Therefore, the vectors described above may be used in immunogenic compositions and vaccines for preventing the development of adenocarcinomas associated with aberrant CEA expression and/or for treating existing cancers. The vectors of the present invention allow for vaccine development and commercialization by eliminating difficulties with obtaining high expression levels of exogenous CEA in successfully transformed host organisms. To this end, one aspect of the instant invention is a method of preventing or treating cancer comprising administering to a mammal a vaccine vector comprising a synthetic codon-optimized nucleic acid molecule, the synthetic codon-optimized nucleic acid molecule comprising a sequence of nucleotides that encodes a rhesus monkey CEA protein as set forth in SEQ ID NO:2 or SEQ ID NO:3.

In accordance with the method described above, the vaccine vector may be administered for the treatment or prevention of cancer in any mammal. In a preferred embodiment of the invention, the mammal is a human.

Further, one of skill in the art may choose any type of vector for use in the treatment and prevention method described. Preferably, the vector is an adenovirus vector or a plasmid vector. In a preferred embodiment of the invention, the vector is an adenoviral vector comprising an adenoviral genome with a deletion in the adenovirus E1 region, and an insert in the adenovirus E1 region, wherein the insert comprises an expression cassette comprising: (a) a synthetic codon-optimized polynucleotide encoding a rhesus monkey CEA protein; and (b) a promoter operably linked to the polynucleotide.

The instant invention further relates to an adenovirus vaccine vector comprising an adenoviral genome with a deletion in the E1 region, and an insert in the E1 region, wherein the insert comprises an expression cassette comprising: (a) a synthetic codon-optimized polynucleotide encoding a rhesus monkey CEA protein, wherein the polynucleotide is codon-optimized for high expression in a human cell; and (b) a promoter operably linked to the polynucleotide.

In a preferred embodiment of this aspect of the invention, the adenovirus vector is an Ad 5 vector.

In another preferred embodiment of the invention, the adenovirus vector is an Ad 6 vector.

In yet another preferred embodiment, the adenovirus vector is an Ad 24 vector.

In another aspect, the invention relates to a vaccine plasmid comprising a plasmid portion and an expression cassette portion, the expression cassette portion comprising: (a) a synthetic codon-optimized polynucleotide encoding a rhesus monkey CEA protein, wherein the polynucleotide is codon-optimized for high level expression in a human cell; and (b) a promoter operably linked to the polynucleotide.

In some embodiments of this invention, the recombinant adenovirus vaccines disclosed herein are used in various prime/boost combinations with a plasmid-based polynucleotide vaccine in order to induce an enhanced immune response. In this case, the two vectors are administered in a “prime and boost” regimen. For example the first type of vector is administered, then after a predetermined amount of time, for example, 2 weeks, 1 month, 2 months, six months, or other appropriate interval, a second type of vector is administered. Preferably the vectors carry expression cassettes encoding the same polynucleotide or combination of polynucleotides. In the embodiment where a plasmid DNA is also used, it is preferred that the vector contain one or more promoters recognized by mammalian or insect cells. In a preferred embodiment, the plasmid would contain a strong promoter such as, but not limited to, the CMV promoter. The synthetic rhesus monkey CEA gene or other gene to be expressed would be linked to such a promoter. An example of such a plasmid would be the mammalian expression plasmid V1Jns as described (J. Shiver et. al. in DNA Vaccines, M. Liu et al. eds., N.Y. Acad. Sci., N.Y., 772:198-208 (1996), which is herein incorporated by reference).

As stated above, an adenoviral vector vaccine and a plasmid vaccine may be administered to a vertebrate as part of a single therapeutic regime to induce an immune response. To this end, the present invention relates to a method of protecting a reammal from cancer comprising: (a) introducing into the mammal a first vector comprising: i) a synthetic codon-optimized polynucleotide encoding a rhesus monkey CEA protein; and ii) a promoter operably linked to the polynucleotide; (b) allowing a predetermined amount of time to pass; and (c) introducing into the mammal a second vector comprising: i) a synthetic codon-optimized polynucleotide encoding a rhesus monkey CEA protein; and ii) a promoter operably linked to the polynucleotide.

In one embodiment of the method of protection described above, the first vector is a plasmid and the second vector is an adenovirus vector. In an alternative embodiment, the first vector is an adenovirus vector and the second vector is a plasmid. In yet another embodiment, both the first and the second vectors are adenovirus vectors.

The instant invention further relates to a method of treating a mammal suffering from an adenocarcinoma comprising: (a) introducing into the mammal a first vector comprising: i) a synthetic codon-optimized polynucleotide encoding a rhesus monkey CEA protein; and ii) a promoter operably linked to the polynucleotide; (b) allowing a predetermined amount of time to pass; and (c) introducing into the mammal a second vector comprising: i) a synthetic codon-optimized polynucleotide encoding a rhesus monkey CEA protein; and ii) a promoter operably linked to the polynucleotide.

In one embodiment of the method of treatment described above, the first vector is a plasmid and the second vector is an adenovirus vector. In an alternative embodiment, the first vector is an adenovirus vector and the second vector is a plasmid. In yet another embodiment, both the first and the second vectors are adenovirus vectors.

The amount of expressible DNA or transcribed RNA to be introduced into a vaccine recipient will depend partially on the strength of the promoters used and on the immunogenicity of the expressed gene product. In general, an immunologically or prophylactically effective dose of about 1 ng to 100 mg, and preferably about 10 μg to 300 μg of a plasmid vaccine vector is administered directly into muscle tissue. An effective dose for recombinant adenovirus is approximately 10⁶-10¹² particles and preferably about 10⁷-10¹¹ particles. Subcutaneous injection, intradermnal introduction, impression though the skin, and other modes of administration such as intraperitoneal, intravenous, or inhalation delivery are also contemplated. It is also contemplated that booster vaccinations may be provided. Parenteral administration, such as intravenous, intramuscular, subcutaneous or other means of administration with adjuvants such as interleukin 12 protein, concurrently with or subsequent to parenteral introduction of the vaccine of this invention is also advantageous.

The vaccine vectors of this invention may be naked, i.e., unassociated with any proteins, adjuvants or other agents which impact on the recipient's immune system. In this case, it is desirable for the vaccine vectors to be in a physiologically acceptable solution, such as, but not limited to, sterile saline or sterile buffered saline. Alternatively, it may be advantageous to administer an immunostimulant, such as an adjuvant, cytokine, protein, or other carrier with the vaccines or immunogenic compositions of the present invention. Therefore, this invention includes the use of such imrnunostimulants in conjunction with the compositions and methods of the present invention. An inununostimulant, as used herein, refers to essentially any substance that enhances or potentiates an immune response (antibody and/or cell-mediated) to an exogenous antigen. Said immunostimulants can be administered in the form of DNA or protein. Any of a variety of immunostimulants may be employed in conjunction with the vaccines and immunogenic compositions of the present inventions, including, but not limited to: GM-CSF, IFNα, tetanus toxoid, IL12, B7.1, LFA-3 and ICAM-1. Said immunostimulants are well-known in the art. Agents which assist in the cellular uptake of DNA, such as, but not limited to calcium ion, may also be used. These agents are generally referred to as transfection facilitating reagents and pharmaceutically acceptable carriers. Those of skill in the art will be able to determine the particular immunostimulant or pharmaceutically acceptable carrier as well as the appropriate time and mode of administration.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defmed in the appended claims.

The following examples illustrate, but do not limit the invention.

### EXAMPLE 1

Construction of Codon-Optimized rhCEA

Wild-type rhesus monkey amino acid sequences were deduced from nucleotide sequences isolated from two different rhesus monkeys. To isolate and determine wild-type nucleotide sequences encoding the rhesus monkey CEA protein, nucleotide sequences from the 5′ and 3′ untranslated regions (UTR) of all known members of the human CEA family were aligned to identify highly conserved regions of the CEA DNA. Based on the CEA gene family homologies, degenerate oligonucleotide primers were designed and PCR conditions were optimized to amplify the rhesus CEA cDNA by reverse transcriptase polymerase chain reaction using RNA isolated from colon samples from two different Rhesus monkeys (Macaca Mulatta). Amplified PCR products of about 2100 bp, the expected size for a CEACAM-5 homolog, were independently obtained from both RNA samples and were purified from agarose gel. Partial sequence analysis of both PCR products revealed high homology with human CEACAM-5. The entire gene sequence was obtained by purifying DNA fragments. Comparison of the rhCEA nucleotide sequences obtained from two rhesus monkeys indicated that there were two nucleotide differences, which code for two different proteins. The predicted protein sequences are shown in FIG. 1(B) (SEQ ID NOs:2 and 3).

Based on the predicted amino acid sequence of one of the two rhCEA proteins described (SEQ ID NO:2), a rhesus CEA cDNA was designed that comprises optimal codons for high expression in human cells, using the Vector NTI program algorithm (InforMax, Rockville, Md.). To increase the level of transcription, an optimized Kozak sequence was inserted 5′ to the ATG. Moreover, two consecutive stop codons were inserted downstream of the coding sequence. The gene was synthesized by Bionexus, Inc (Oakland, Calif.) by PCR-mediated oligonucleotide assembly and cloned in the vector pCR-blunt (Invitrogen, Carlsbad, Calif.). To verify the sequence, both strands of the gene were sequenced by using ABI 377 automated sequencer. Autoassembler program was used to compare the sequence data of the synthesized gene with the expected sequence. The open reading frame of the optimized rhesus CEA sequence is depicted in FIG. 1(A) (SEQ ID NO:1).

### EXAMPLE 2

Plasmid Constructs and Adenovirus Generation

pV1J-rhCEAopt: RhCEAopt was excised as an EcoRI fragment from pCR-blunt-rhCEAopt vector and inserted in pV1J-nsA vector, obtaining pV1J-RhCEAopt.

pAd5-rhCEAopt and pAd24-rhCEAopt: For adenovirus 5 generation, pMRK-rhCEAopt was obtained by subcloning rhCEAopt as a HincII/XhoI fragment in SwaI/SalI sites of the polyMRK vector (See Emini et al., WO 02/22080, which is hereby incorporated by reference). For Ad24 generation, the expression cassette was excised from pMRK-rhCEAopt as an SspI/AscI fragment and inserted in the shuttle vector pABS-Ad17-3 in the EcoRI site, thus generating pABS-Ad17-rhCEAopt. A PacI/StuI fragment from pMRK-rhCEAopt and a XhoI/XbaI fragment from pABS-Ad17-rhCEAopt containing the expression cassette for rhCEAopt and E1 flanking Ad5 and Ad17/24 regions respectively, were recombined to ClaI linearized pAd5 or SwaI linearized pAd24 using BJ5183 E. Coli cells. The resulting plasmids were pAd5-rhCEAopt and pAd24-rhCEAopt. These plasmids were cut with PacI or PmeI respectively, to release the adenovirus ITRs and transfected in PerC-6 cells by Lipofectamine 2000 (Life Technologies, Carlsbad, Calif.). The amplification of the vectors was carried out through serial passages. Ad5-rhCEAopt and Ad24rhCEAopt were purified through a standard CsCl purification protocol and extensively dialyzed against A105 buffer (5 mM Tris pH 8.0, 1 mM MgCl₂, 75 mM NaCl, 5% Sucrose, 0.005% Tween20).

pV1J-rhCEA. Ad5-rhCEA and Ad6-rhCEA: RhCEA was excised with PstI/XhoI from pCMV-script EX phagemid vector and inserted in pBluescript H KS vector, obtaining pBS-RhCEA. The insert was entirely sequenced and then subcloned as SmaI/XhoI fragment in pV1JnsA vector, obtaining pV1J-RhCEA. The shuttle plasmid pMRK-RhCEA for adenovirus generation was obtained by subeloning the same fragment in the polyMRK vector. A PacI/StuI fragment from pMRK-RhCEA containing the expression cassette for RhCEA and E1 flanking Ad5 regions was recombined to ClaI linearized pAd5 or pAd6 in BJ5183 E. Coli cells. The resulting plasmids were pAd5-RhCEA and pAd6-RhCEA. Both plasmids were cut with PacI to release the adenovirus ITRs and transfected in PerC-6 cells. Viral amplification was carried out through serial passages. Ad5-RhCEA and Ad6-RhCEA were purified using a standard CsCl purification protocol and extensively dyalized against A105 buffer (5 mM Tris pH 8.0, 1 mM MgC12, 75mM NaCl, 5% sucrose, 0.005% Tween20).

### EXAMPLE 3

In vitro Expression of Rhesus CEA

Rhesus CEA expression from the constructs described above was verified by western blot and FACS analysis. Plasmids were transfected in HeLa cells with Lipofectamine 2000 (Life Technologies) according to manufacturer directions. Adenovirus infections were performed in serum-free medium for 30 min at 37° C., then fresh medium was added to cells. 48 hours later, whole cell lysates were analyzed by western blot using a rabbit polyclonal serum against human CEA (Fitzgerald Industries International Inc., Concord Mass., 1:1500 dilution). Rhesus CEA was detected as a 180-200KDa band.

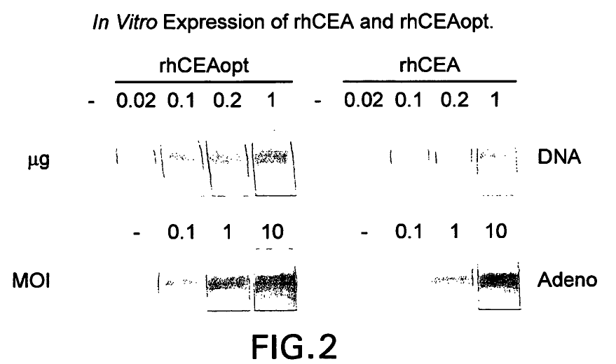

Western blot analysis demonstrated that transfection of HeLa cells with an expression plasmid (pV1J-rhCEAopt) carrying the optimized rhesus CEA cDNA (rhCEAopt) at different doses showed 100 fold greater protein levels than a similar vector carrying the native cDNA (pV1J-rhCEAopt). See FIG. 2. Similarly, infection of HeLa cells with a first generation ΔE1-ΔE3 adenovirus vector expressing rhCEAopt (MRKAd5-rhCEAopt) showed a significant improvement in expression level (FIG. 2). Thus, the optimization of the rhesus CEA coding sequence effectively enhanced the level of expression of rhCEA in vitro.

### EXAMPLE 4

Mice Immunization

C57BL/6 mice (H-2^{b}) were purchased by Charles River (Lecco, Italy). CEA transgenic (CEA.tg) mice (H-2^{b}) were provided by J. Prirnus (Vanderbilt University) and kept in standard conditions.

For electro gene transfer (EGT), mice quadriceps were either surgically exposed or directly injected with the indicated doses of pV1J-rhCEA or pV1J-rhCEAopt and electrically stimulated as previously described (Rizzuto at al. Proc. Natl. Acad. Sci U.S.A. 96(11): 6417-22 (1999)).

For adenovirus injection, Ad5-rhCEA, Ad6-rhCEA, Ad5-rhCEAopt or Ad24-rhCEAopt were injected in mice quadriceps at the indicated doses.

### EXAMPLE 5

Rhesus CEA Ouantitation and Antibody Titration

Sera for antibody titration were obtained by retro-orbital bleeding. For rhesus CEA measurement in the blood, Elisa plates (Nunc maxisorp) were coated O/N at 4° C. with a polyclonal anti rhCEA mouse serum in carbonate buffer (50 mM NaHCO₃ pH 9.4). Plates were then blocked with PBS containing 5% BSA for 1 hr at 37° C. Cell supernatants or sera were then diluted in PBS 5% BSA and incubated for 2 hr at RT. After washing 5 times with PBS/0.05% Tween 20, anti-CEA rabbit polyclonal antibody was added at 1:2000 dilution and incubated for further 2 hr. Plates were then washed again and detecting antibody anti-rabbit IgG-AP conjugated was added at 1:2000 dilution for 1 hr at RT. Final detection was done with 100 μl/well p-nitrophenyl phosphate disodium, 1.0 mg/ml in 10% diethanolamine buffer, pH 9.8 containing 0.5 mm MgCl2 and reading at OD₄₀₅.

For antibody titration, Elisa plates were coated with 100 ng/well CEA protein (Fitzgerald, highly pure CEA), diluted in coating buffer and incubated O/N at 4° C. Mouse sera were diluted in PBS 5% BSA. Pre-immune sera were used as background. Diluted sera were incubated O/N at 4° C. Washes were carried out with PBS, 1% BSA, 0.05% tween 20. Detecting antibody (goat anti-mouse IgG Peroxidase, Sigma), was diluted 1/2000 in PBS, 5%BSA and incubated for 2-3 hr at RT on a shaker. After washing, plates were developed with 100 μl/well of TMB substrate (Pierce Biotechnology, Inc., Rockford, Ill.). The reaction was stopped with 25 μl/well of 1M H₂SO₄ solution and plates were read at 450 nm/620 nm. Anti-CEA serum titers were calculated as the limiting dilution of serum producing an absorbance at least 3-fold greater than the absorbance of autologous pre-immune serum at the same dilution.

### EXAMPLE 6

ELISPOT Assay for IFN-γ

96-well MAIP plates (Millipore) were coated with purified rat anti-mouse IFN-γ (IgG1, clone R4-6A2, Pharmingen) at 2.5 μg/ml in sterile PBS, aliquoted at 100 μl per well. After washing with sterile PBS, blocking of plates was done with 200 μl per well of R10 medium at 37° C. for at least 2 hours.

For preparation of splenocytes, spleens were removed from sacrificed mice in a sterile manner and disrupted by scratching through a grid. Red blood cell osmotic lysis was obtained by adding 1 ml of 0.1 ×PBS to cell pellet and vortexing for no more than 15 s. 1 ml of 2×PBS was then added and the volume was brought up to 4 ml with PBS 1×. After spinning at 1200 rpm for 10 minutes at RT, the cell pellet was resuspended in 1 ml of R10 medium and viable cells were counted.

Splenocytes were plated at 5×10⁵ and 2.5×10⁵/well with 1 μg/ml each peptide in R10 and incubated for 16-20h in a CO₂ incubator at 37° C. Concanavalin A (ConA) at 5 μg/ml was used as positive internal control for each mouse. After washing with PBS, 0.05% Tween 20, plates were incubated O/N at 4° C. with 50 μl/well of biotin-conjugated rat anti-mouse IFN-γ (Rat IgG1, clone XMG 1.2, PharMingen), diluted 1:250 in assay buffer (PBS-5%FBS-0.005%Tween-20). The next day, plates were washed and incubated for 2 h at RT with streptavidin-AP conjugate (Pharmingen, San Jose, Calif.) diluted 1:2500 in assay buffer. After extensive washing, plates were developed by addition of 50 μl/well NBT/B-CIP (Pierce) until development of spots was observed through the microscope. The reaction was stopped by washing plates thoroughly with distilled water. Plates were allowed to air-dry completely, and spots were counted using an automated ELISPOT reader.

### EXAMPLE 7

Intracellular Staining for IFN-γ

For PBMC preparation, about 200 μl blood were obtained from each mouse by retro-orbital bleeding and heparinized. Erythrocyte lysis was obtained by incubation for 10 min with ACK lysing buffer (Life Technologies). After centrifugation, white cells were resuspended in R10 medium. 1-2×10⁶ splenocytes or PBMC were resuspended in 1 ml R10. Antigen peptides or peptide pools were added to a final concentration of 1 μg/ml with Brefeldin A.

After 12 hours incubation at 37° C., cells were washed with 3 ml FACS buffer (PBS, 1% FCS) and centrifuged for 10 min at RT. Incubation with anti-mouse CD16/CD32 was carried out in 100 μl FACS buffer for 15 min at 4° C. After washing, for the surface antigen staining, APC conjugated anti-mouse CD3 ε, PE conjugated anti-mouse CD4, PerCP conjugated anti-mouse CD8 α all diluted 1:50 in FACS buffer were added in 100 μl final volume and incubated for 30 min at RT in the dark. After washing with PermWash (Pharmingen), cells were resuspended in 100 μl of Cytofix-Cytoperm solution (Pharmningen), vortexed and incubated for 20 min at 4° C. in the dark. For intracellular staining, cells were incubated with FITC conjugated anti-mouse interferon-γ diluted 1:50 in PermWash (100 μl final volume) for 30 min at RT in the dark After washing, cells were resuspended in 250-300 μl 1% formaldehyde in PBS and analyzed with a FACScalibur (Becton Dickinson, San Jose, Calif.).

### EXAMPLE 8

Expression and Immunization Studies in Wild-Type Mice

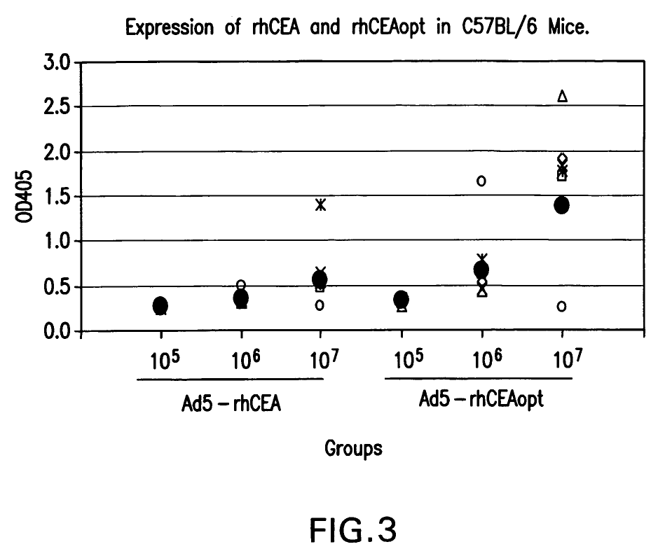

Transfection of HeLa cells with constructs carrying wild-type or codon-optimized rhesus CEA demonstrated that optimization of the rhesus CEA coding sequence enhanced the level of expression of rhCEA in vitro. See EXAMPLE 3. To determine whether a higher level of expression could be measured also in vivo, C57BL/6 mice were either injected intramuscularly with pV1J-rhCEAopt followed by electro gene transfer (EGT) or with MRKAd5-rhCEAopt and compared with similar vectors carrying wild type CEA. Four days later, mice were bled and circulating levels of rhCEA were measured by ELISA. Greater protein levels were obtained in Ad5-rhCEAopt injected mice at different doses than those detected upon injection of vectors encoding the wt rhCEA (FIG. 3). No significant expression of rhesus CEA could be measured upon DNA plasmid injection (data not shown).

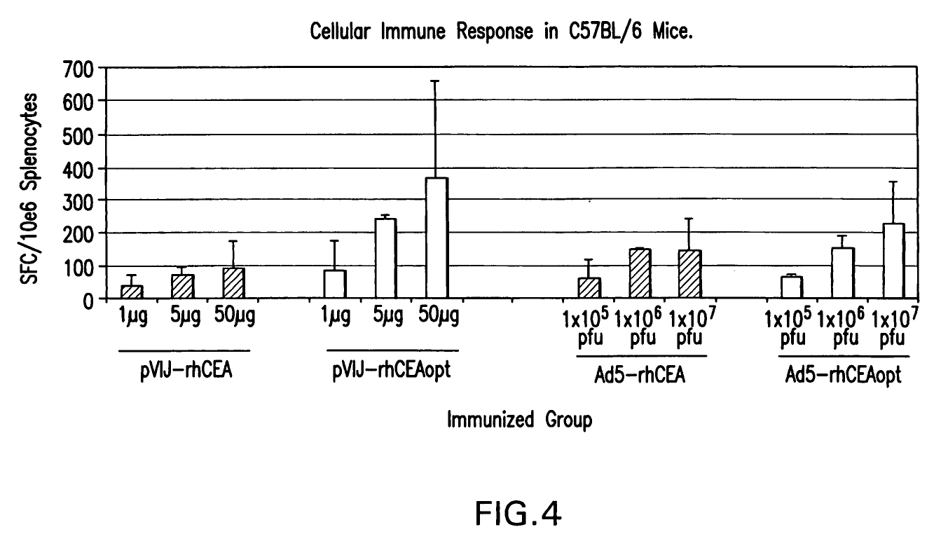

To verify whether the higher expression was related to a higher immunogenicity, cellular immune response in injected mice was measured by ELISPOT using rhCEA peptides as stimulators. Significant enhancement of the immune response could be measured using rhCEAopt expressing DNA vectors compared to rhCEA vectors (FIG. 4). These data show that optimization of rhCEA cDNA was effective in augmenting the level of expression of rhesus CEA in vivo and, more importantly, in enhancing cell mediated immune response in wild type C57BL/6 mice.

### EXAMPLE 9

Mixed Modality Vaccination Studies in CEA Transgenic Mice

Immunization experiments with wild-type rhesus monkey CEA demonstrated the ability of rhCEA xenogeneic vaccination to elicit an immune response against human CEA as self-antigen. Briefly, CEA transgenic (CEA.Tg) mice were immunized with vectors encoding human (self) or rhesus CEA (xeno) and breakage of immune tolerance was achieved using rhCEA bearing vectors. CEA.Tg mice are transgenic mice that express human CEA as a self-antigen with a tissue distribution similar to that of humans.

To demonstrate if rhCEAopt expression vectors could better break the immune tolerance to human CEA, CEA.Tg mice were immunized with DNA plasmids followed by EGT. Mice were injected 4 times at weekly intervals with pV1J vector expressing either hCEA, hCEAopt, rhCEA or rhCEAopt. Moreover, to stimulate the immune response against the xeno-antigen and then direct it to the self antigen to break the immune tolerance, one group received 4 injections of a 50% mix containing hCEAopt/rhCEAopt vectors and a second group was treated with 3 injections of pV1J-rhCEAopt and a fourth injection of pV1J-hCEAopt.

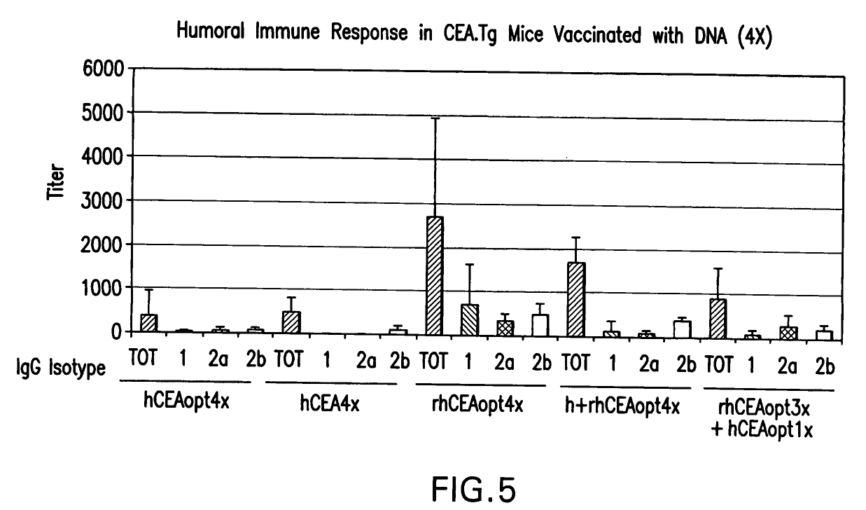

No detectable cell-mediated immune response could be measured in any experimental group after 4 DNA injections as measured by ICS from PBMC (data not shown). Unlike the cellular immune response, an enhanced humoral immune response to hCEA was detected in CEA.Tg mice vaccinated with rhCEAopt. Antibody measurements obtained by ELISA assay demonstrated a 10-fold increase in the group treated with rhCEAopt compared to the hCEAopt immunized group (FIG. 5). Consistent with this result, significant IgG titer (>1:1000) was obtained only when rhCEAopt was present in the immunization schedule. The anti-CEA Ab titer in mice immunized with wild-type rhesus CEA was not significantly different from the Ab titer in mice treated with hCEAopt (data not shown).

IgG isotype titer was also measured as a quality of the response. An increased IgG1/IgG2a ratio was obtained in mice immunized with rhCEAopt relative to those mice vaccinated with the hCEA and hCEAopt expressing vectors, which suggests that the xeno-gene elicited an enhanced Th2-type immune response.

Each group of mice was then boosted with an Ad5 vector expressing the transgene of the fourth DNA injection. Thus, mice immunized with the pV1J mix described above received a 50% mix of Ad5-rhCEAopt and Ad5-hCEAopt as a booster. Similarly, mice vaccinated with 3 injections of rhCEAopt followed by one of pV1J-hCEAopt were boosted with Ad5-hCEAopt.

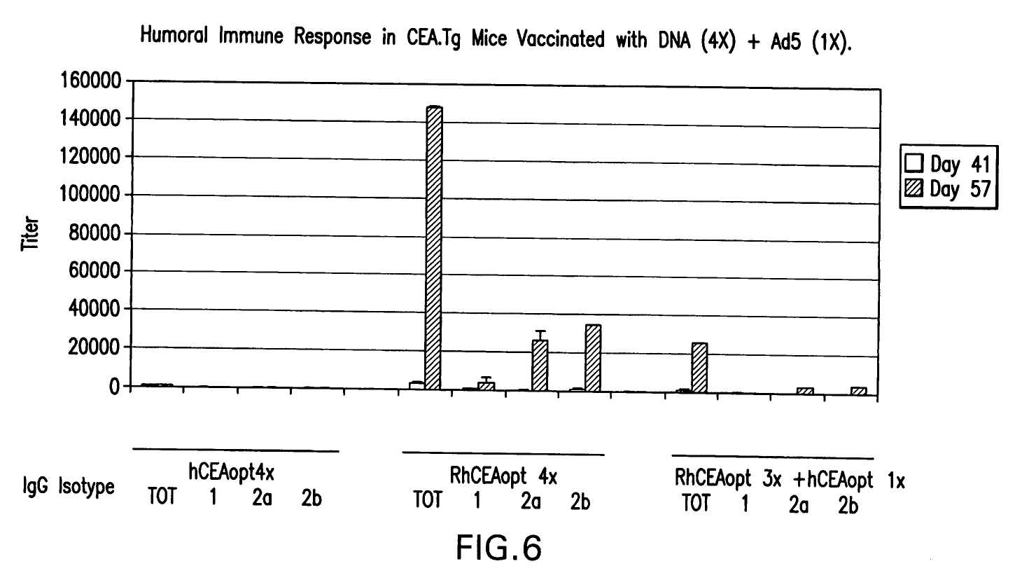

Sixteen days after the booster injection, the humoral immune response was measured by ELISA assay. Results demonstrate that the Ad5 boost greatly enhanced the antibody titer in rhCEAopt immunized mice (average 1:150000), but not significantly in the other groups (FIG. 6). Thus, the use of rhCEAopt as immunogen induces high cross reactive human CEA-specific antibody response—300—fold higher than hCEA containing vectors.

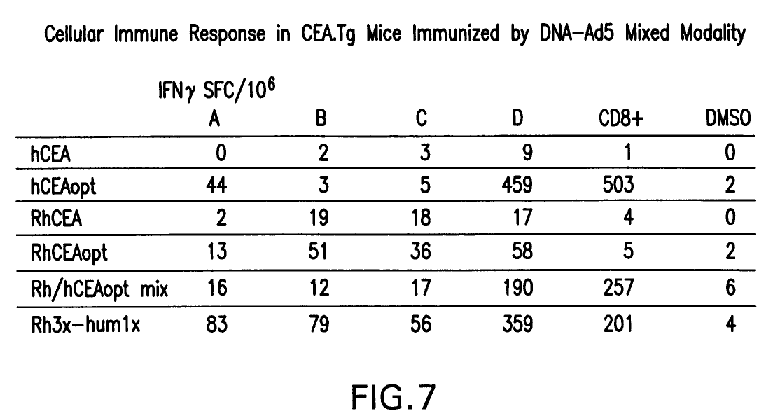

To verify whether the cell mediated immune response against human CEA was elicited in the above-described groups of mice after Ad5 boosting, an ELISPOT IFNγ assay was performed after stimulation with human CEA specific peptides. 15mer peptides covering the entire human CEA protein, divided into four sections, were assembled, generating pools A, B, C and D. Results demonstrate that optimization of the CEA nucleotide sequence increased the immune response to human CEA (FIG. 7). In fact, both hCEAopt and rhCEAopt increased the number of measured effectors. Interestingly, immunization with rhCEAopt alone or in combination with hCEAopt changed the quality and distribution of the cell mediated response along the human CEA protein. Additionally, increased SFC were measured with pools B and C as stimulators in these groups compared to hCEAopt immunized mice.

### EXAMPLE 10

Identification of Immunogenic Peptides

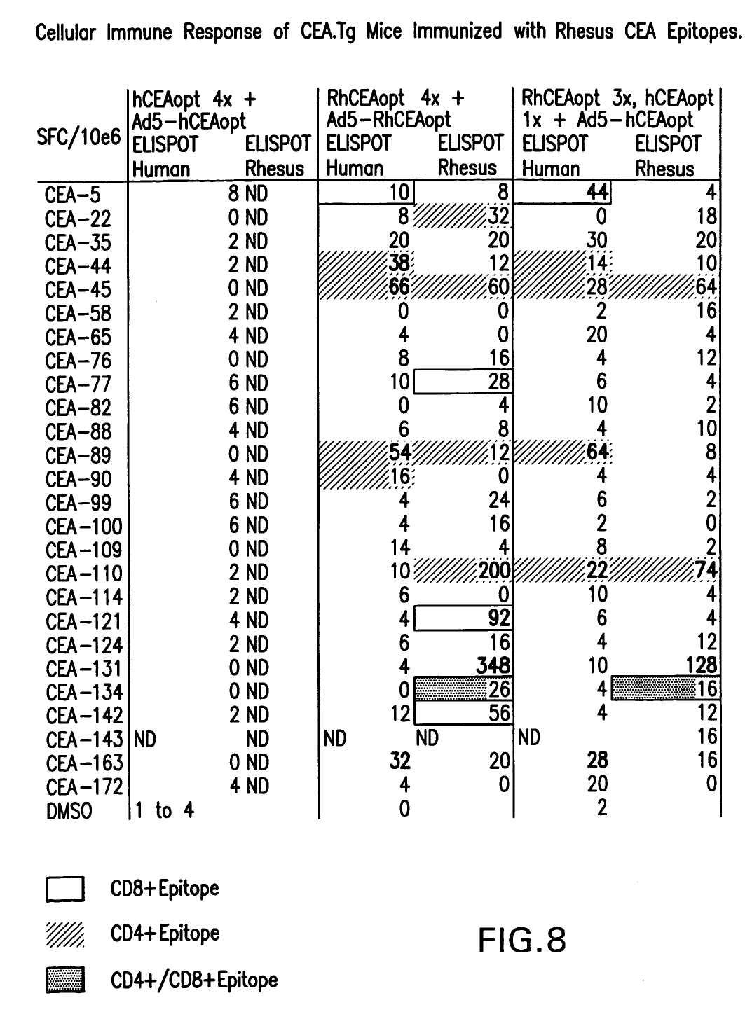

To identify immunogenic regions of the human and rhesus CEA proteins, an ELISPOT assay was performed using individually peptides spanning the two proteins. Responsive peptides identified by ICS in the rhesus CEA protein were significantly different than those identified in the human protein (FIG. 8). Some responses were comparable or even higher against human peptides than against the corresponding region of rhesus protein when rhCEAopt was included in the immunization protocol.

For the human CEA protein, CD4+ (CEA44 (a.a. 173 to a.a. 187)−CEA45 (a.a.177 to a.a. 191); CEA89 (a.a. 353 -a.a. 367)−CEA90 (a.a. 357 - a.a. 371); and CEA 1l0( a.a. 437 to a.a. 451)) and CD8+ (CEA5(aa.17 -a.a.31)) epitopes were induced by rhCEAopt or rhCEAopt/hCEAopt sequential immunizations. This enhanced CD4+ response compared to hCEAopt immunized group could be related to the increased antibody titer measured in rhCEAopt immunized mice.

For the rhesus CEA protein, specific CD4+ responses were measured with CEA22 (a.a.85-a.a.99) and CEA110 (a.a.437-a.a.451), while CEA77 (a.a.305-a.a.319), CEA121 (a.a.481-a.a.495) and CEA142 (a.a.565-a.a.579) were strong CD8+ epitopes. Interestingly, a peptide able to activate both CD4+ and CD8+ IFNγ secretion was identified (CEA134 (a.a.533-a.a.547). These results show that rhCEAopt used as xeno-antigen induced a strong and qualitatively different cross reactive immnune response to human CEA.

### EXAMPLE 11

Expression and Immunization with Different Adenovirus Serotypes

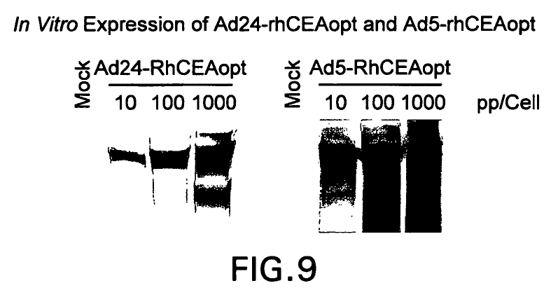

To test alternative Ad serotypes as boosters of the immune response, Ad24-rhCEAopt, which comprises an Ad24 vector expressing codon-optimized rhesus CEA (see EXAMPLE 2), was constructed as a potential booster of the immune response elicited in AdS injected animals. Rhesus CEA expression was verified in vitro and compared to AdS-rhCEAopt (FIG. 9). A 50-100 fold lower protein level was obtained with Ad24 infection. This observation could be due to a different ratio of physical/transducing particles between Ad5 and Ad24 serotypes.

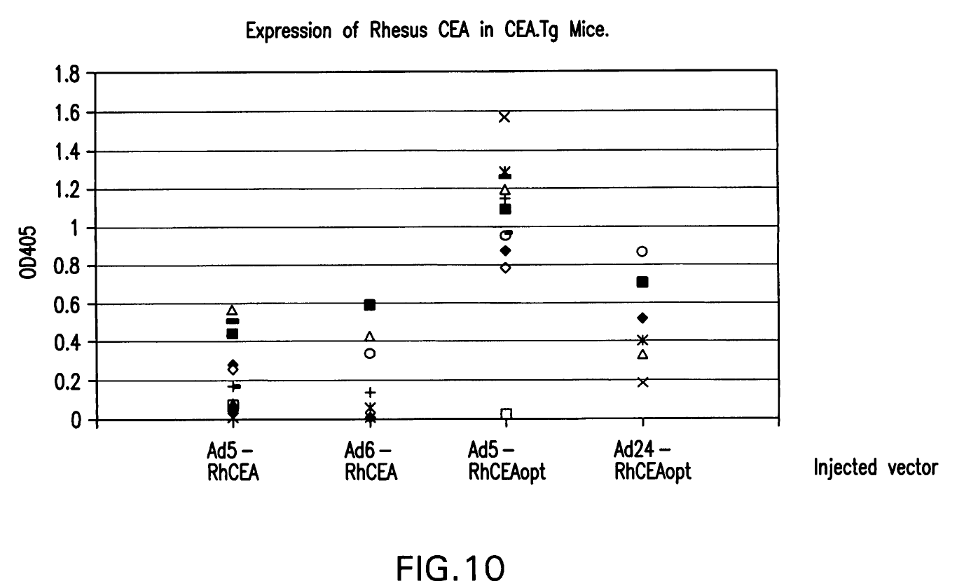

To verify the level of expression in vivo, CEA.Tg mice were injected with adeno vectors expressing either rhCEA or rhCEAopt (FIG. 10). The systemic expression of both AdS-rhCEAopt and Ad24-rhCEAopt was detectably higher than expression of Ad5 or Ad6 vectors comprising wild-type gene. According to in vitro data, Ad24 mediated expression was lower than Ad5.

The immunogenicity of Ad24 was then tested in CEA.Tg mice. Immune responses elicited by the following prime- boost modalities were compared: Ad5- Ad24, Ad24- Ad24 and Ad5-Ad5. Fourteen days after the boost, peripheral cell mediated immune response was measured by ICS.

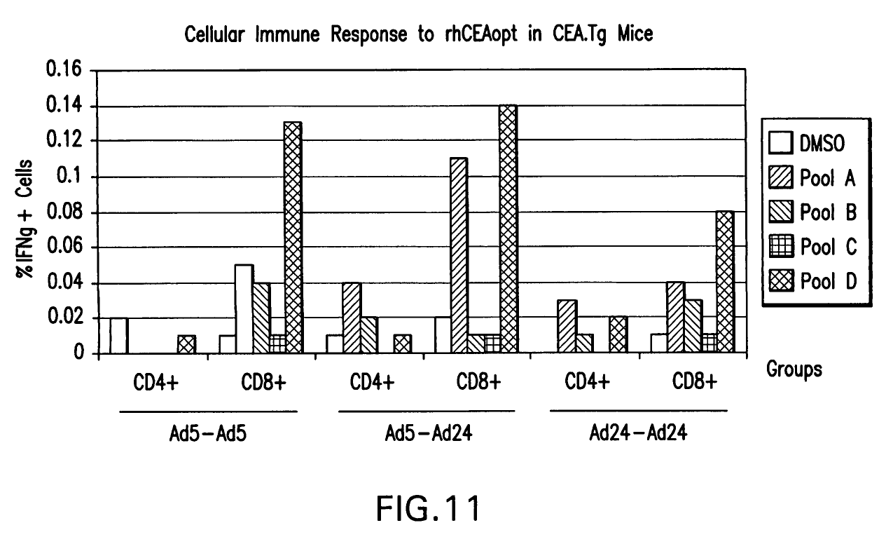

The results indicate that the Ad5-Ad5 protocol elicited a significantly higher cell mediated inmnune response against different CEA peptide pools than the Ad24-Ad24 protocol (FIG. 11). Interestingly, the Ad5-Ad24 protocol elicited a response comparable to Ad5-Ad5. These experiments demonstrate that Ad24 is a good booster of the immune response elicited by other Ad serotypes.

### EXAMPLE 12

Immunization of Rhesus Macagues with rhCEAopt: Protocol CV-1

In order to assess the efficiency of immunization of rhesus macaques (macaca mulatta) with the optimized version of rhesus CEA, immunization studies were performed at the Biomedical Primate Research Centre (BPRC, Rijswijk, The Netherlands). Such immunization studies were designed to evaluate both B and T cell responses to immunization with the rhesus CEA antigen.

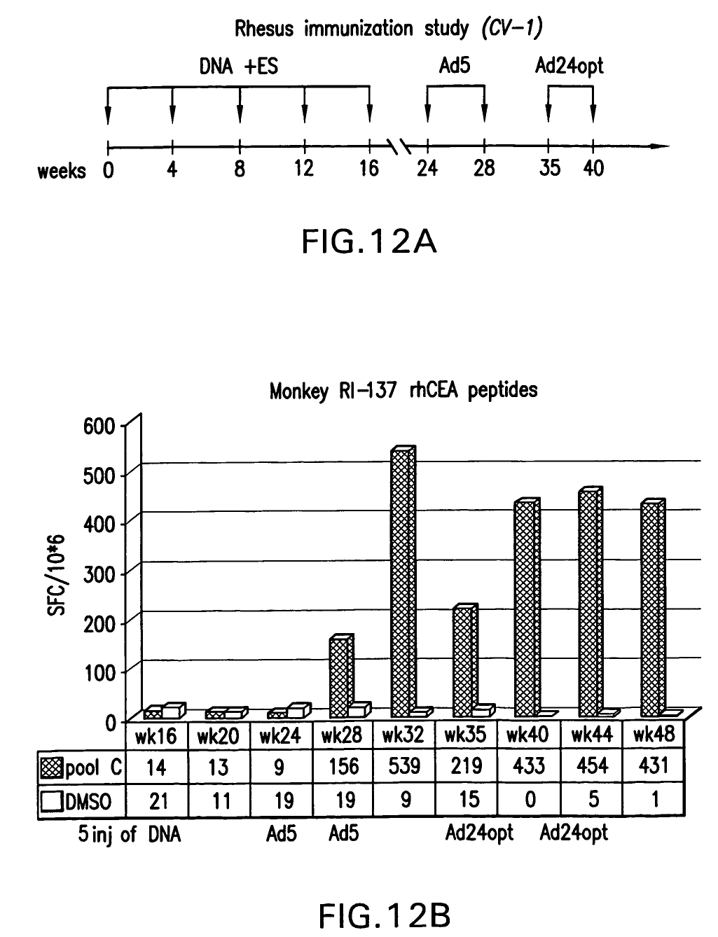

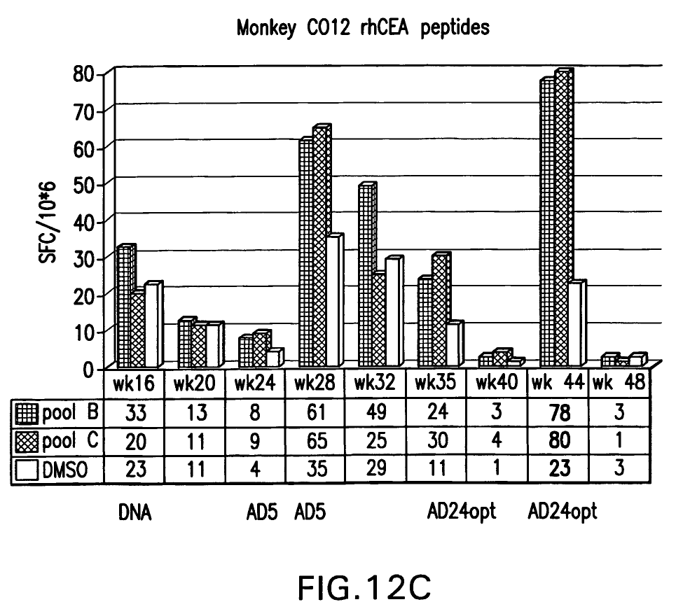

In a first study (CV-1, FIG. 12), 1 group of monkeys (consisting of 2 males and 2 females) was immunized with a plasmid DNA vector or adenovirus vector expressing the wild-type or optimized version of rhesus CEACAM-5. For priming, animals were vaccinated intramuscularly with plasmid DNA expressing wild-type rhCEA at weeks 0, 4, 8, 12, 16, and 24 by injection of DNA followed by electrical stimulation. The DNA injection consisted of a I ml solution (split over 2 sites with 0.5 ml/site) containing 5 mg plasmid DNA for animals weighing 2-5 kilos. Animals were injected under anesthesia (mixture of ketamine/xylazine).

For electrostimulation, 2 trains of 100 square bipolar pulses (1 sec each), were delivered every other second for a total treatment time of 3 sec. The pulse length was 2 msec/phase with a pulse frequency and amplitude of 100 Hz and 100 mA (constant current mode), respectively.

To measure the immune response to CEA using the above immunization protocol, blood samples were collected every four weeks for a total duration of one year. The humoral response was measured by ELISA assay and the cell mediated response was measured by IFNγ Elispot assay (FIG. 12B). Since no significant immune response was obtained at week 16, two further injections (week 24 and 28) were carried out using Ad5 expressing the wild-type version of the tumour antigen. Upon Ad5 injection, the immune response against rhCEA could be measured for two monkeys (RI137 and CO12) covering peptide pool C and pool B+C, respectively. At week 35, the immune response started to decline in both monkeys. To maintain the immune response, Ad24 expressing the optimized version of rhCEA was injected at weeks 35 and 40, resulting in a measurable boosting effect in monkeys RI137 and CO12 at weeks 40 and 44, respectively.

These data show that adenovirus vectors are effective in inducing a specific immune response to rhCEA in rhesus monkeys after priming with DNA. These results further indicate that Ad24-rhCEAopt is a good vaccine booster.

### EXAMPLE 13

Immunization of Rhesus Macaques with rhCEAopt: Protocol CV-2

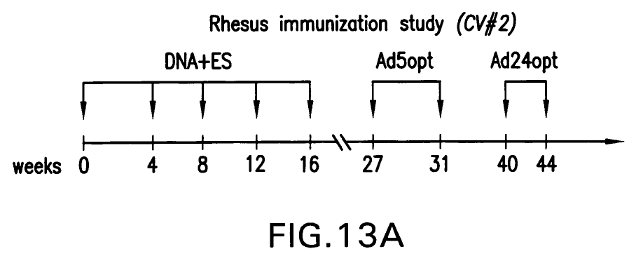

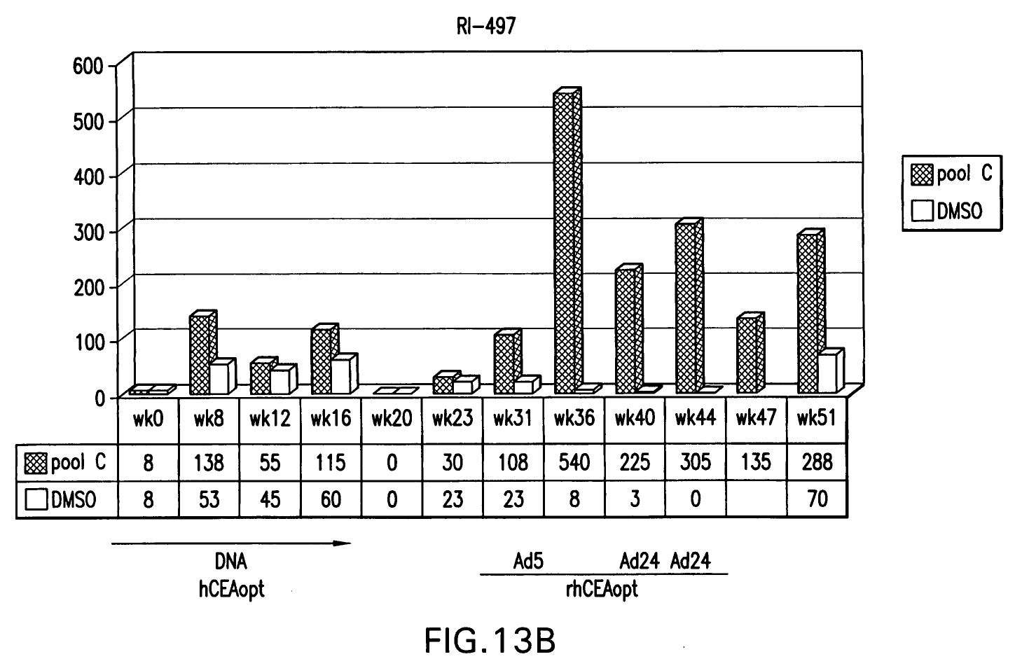

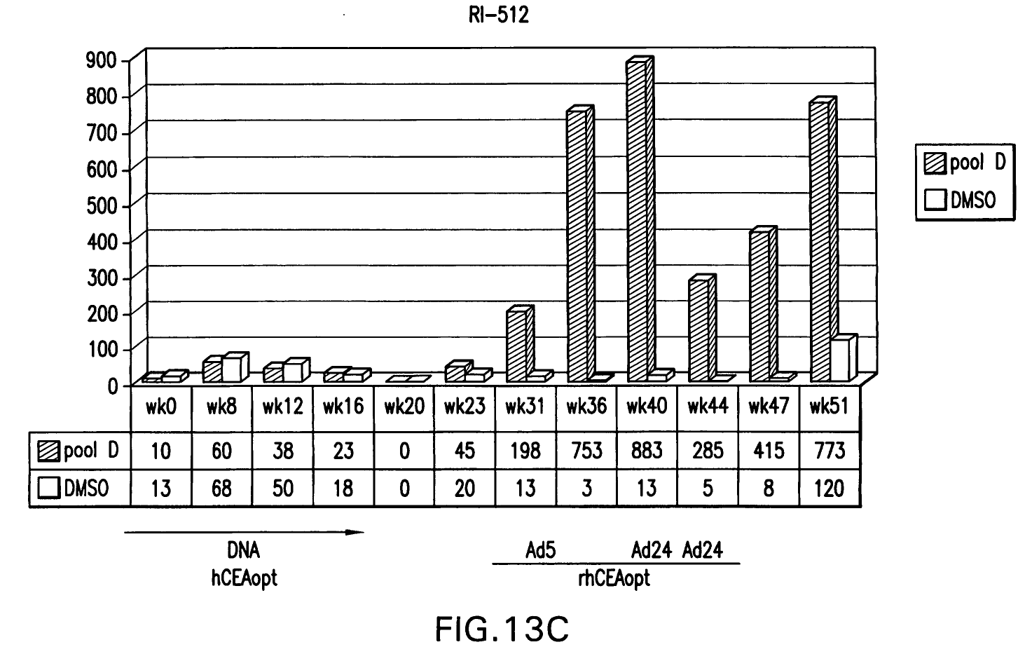

A second series of immunization studies (CV-2, FIG. 13) was performed in order to assess the efficiency of immunization of Rhesus macaques (macaca mulatta) with the rhesus homologues of the human tumour antigens HER2/neu, Ep-CAM and CEA, which are all expressed in colorectal carcinomas. Protocols were designed to evaluate both B and T cell responses to these tumor antigens in combination.

In this study, a group of 4 rhesus monkeys was immunized with a mixture of plasmid DNA vectors expressing the human tumour-associated antigens (TAAs) Ep-CAM, CEA, and HER2/neu as xeno-antigens. For priming, animals were vaccinated intramuscularly with injections at weeks 0, 4, 8, 12 and l6 followed by electrostimulation. The DNA injection consisted of a I ml solution (split over 2 sites with 0.5 ml/site) containing 6 mg plasmid DNA for animals weighing 2-5 kilos. Subsequently, monkeys received two injections of Ad5 (weeks 27+31) followed by two injections of Ad24 (weeks40+44) expressing the optimized versions of the three TAAs. Each injection comprised a mnixture of 1×10e11pp of Ad-rhCEAopt, 1×10e11 of Ad-rhEpCAMopt and 1×10-e11 of Ad-rhHER2opt, serotype 5 or 24.

Breakage of immune tolerance was obtained in two monkeys (RI497 and RI512, FIG. 13(B)) when Ad5 injection was used as booster and maintained by Ad24 successive injections. To determine if this immune response will be maintained for longer periods of time, the CV-2 protocol is followed for additional weeks and the resulting immune response is measured every four weeks.

The results of the CV-2 protocol show that adenoviral vectors can be used to induce and maintain an effective immune response against rhesus CEA and, importantly, that this immune response can be achieved in the context of a multiple vaccination schedule.

### EXAMPLE 14

Immunization of Rhesus Macaques with rhCEAopt: Protocol CV-3

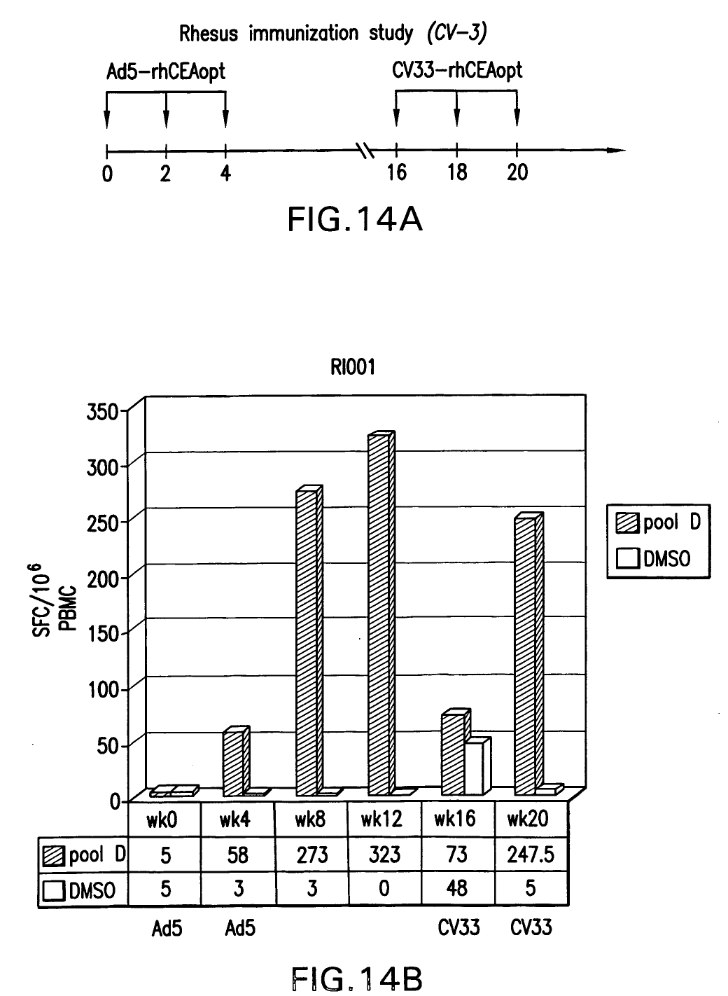

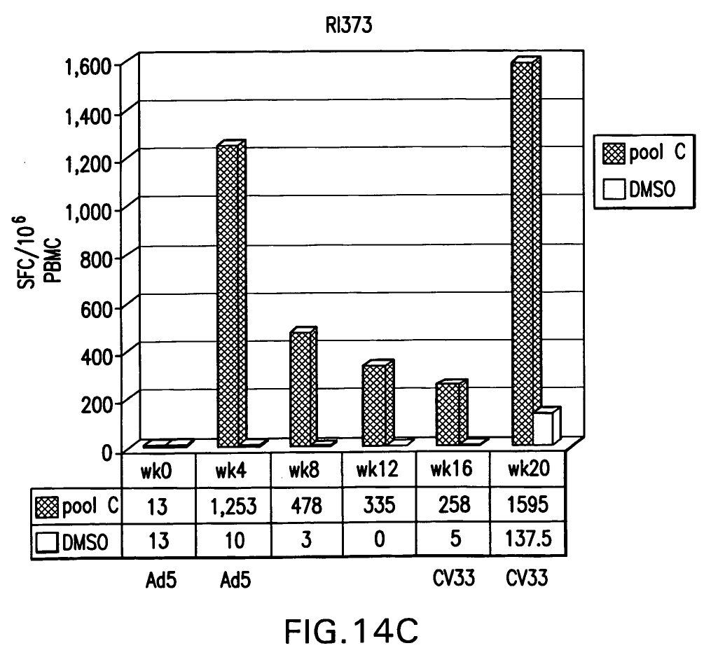

To determine if an effective immune response against rhCEA can be obtained using only adenovirus vectors to prime and boost, a third study, based exclusively on adenoviral vectors was designed (Protocol CV-3, FIG. 14). In comparison with the previous protocols, the number of injections for each virus increased from two to three at 0, 2 and 4 weeks for Ad5. additionally, CV33 injections were given at 16, 18 and 20 weeks.

Similar to the CV-2 protocol (EXAMPLE 13), 4 monkeys were immunized simultaneously with optimized rhCEA, rhEpCAM and rhHER2 expressing vectors. Consistent breakage of immune tolerance to rhCEA was measured at week 4 in two monkeys (RI001 and RI373, FIG. 14(B)). This response was efficiently boosted by a different Ad serotype (CV33) expressing the optimized tumor antigens.

To determine if this immune response is maintained for longer periods of time, the CV-3 protocol is followed for additional weeks and the resulting immune response is measured every four weeks.

These data show that adenoviral vectors are sufficient to break the immune tolerance to rhCEA and maintain the response over time in a rhesus monkey animal model.

1. A synthetic nucleic acid molecule comprising a sequence of nucleotides that encodes a rhesus monkey carcinoembryonic antigen (CEA) protein as set forth in SEQ ID NO:2 or SEQ ID NO:3, the synthetic nucleic acid molecule being codon-optimized for high level expression in a human cell.

2-5. (canceled)

6. The synthetic nucleic acid molecule of claim 1 wherein the sequence of nucleotides comprises the sequence of nucleotides set forth in SEQ ID NO: 1.

7. A vector comprising the nucleic acid molecule of claim 1.

8. A host cell comprising the vector of claim 7.

9. A process for expressing a rhesus monkey carcinoembryonic antigen (CEA) protein in a recombinant host cell, comprising:
(a) introducing a vector comprising the nucleic acid of claim 1 into a suitable host cell; and, (b) culturing the host cell under conditions which allow expression of said rhesus monkey CEA protein.

10. A method of preventing or treating cancer
comprising administering to a human a vaccine vector comprising the synthetic nucleic acid molecule of claim 1,

11. (canceled)

12. A method according to claim 10 wherein the vector is an adenovirus vector or a plasmid vector.

13-14. (canceled)

15. An adenovirus vaccine vector comprising an adenoviral genome with a deletion in the E1 region, and an insert in the E1 region, wherein the insert comprises an expression cassette comprising:
(a) a polynucleotide encoding a rhesus monkey carcinoembrvonic antigen (CEA) protein as set forth in SEO ID NO:2 or SEQ ID NO:3 the polynucleotide being codon-optimized for high-level expression in a human cell; and
(b) a promoter operably linked to the polynucleotide.

16. An adenovirus vector according to claim 15 wherein the adenoviral genome is selected from the g:roup consisting of: Ad5, Ad6 and Ad24.

17-18. (canceled)

19. A vaccine plasmid comprising a plasmid portion and an expression cassette portion, the expression cassette portion comprising:
(a) a polynucleotide encoding a rhesus monkey carcinoembryonic antigen (CEA) protein as set forth in SEQ ID NO:2 or SEQ ID NO:3, the polynucleotide being codon-optimized for high-level expression in a human cell; and
(b) a promoter operably linked to the polynucleotide.

20. A method of protecting a mammal from cancer comprising:
(a) introducing into the mammal a first vector comprising:
(i) a codon-optimized polynucleotide encoding a rhesus monkey carcinoembryonic antigen (CEA) protein; and
(ii) a promoter operably linked to the polynucleotide;
(b) allowing a predetermined amount of time to pass; and
(c) introducing into the mammal a second vector comprising:
(i) a codon-optimized polynucleotide encoding a rhesus monkey CEA protein; and
(ii) a promoter operably linked to the polynucleotide.

21. A method according to claim 20 wherein the first vector is a plasmid and the second vector is an adenovirus vector.

22. A method according to claim 20 wherein the first vector is an adenovirus vector and the second vector is a plasmid.

23-29. (canceled)
